(19) United States
(12) Patent Application Publication
(10) Pub. No.: US 2016/0192976 A1
(43) Pub. Date: Jul. 7, 2016

(54) SYSTEMS, DEVICES, APPARATUS AND METHOD DEVICES FOR PROVIDING ENDOSCOPIC MUCOSAL THERAPY

(71) Applicant: THE GENERAL HOSPITAL CORPORATION, Boston, MA (US)

(72) Inventor: Benjamin VAKOC, Arlington, MA (US)

(21) Appl. No.: 14/916,684

(22) PCT Filed: Sep. 5, 2014
(86) PCT No.: PCT/US14/54334

§ 371 Date: Mar. 4, 2016

Related U.S. Application Data
(60) Provisional application No. 61/874,686, filed on Sep. 6, 2013.

(51) Int. Cl. A61B 18/04; A61B 17/00; A61B 18/14; A61N 7/00

(52) U.S. Cl. CPC A61B 18/04; A61N 7/00; A61B 17/00234; A61B 18/1492; A61B 2018/00291

(57) ABSTRACT

Apparatus and method for effecting at least one first layer of at least one biological structure of an internal organ can be provided. For example, it is possible to utilize at least one structural arrangement which is configured to be inserted into or adjacent to the organ, and including at least one vacuum device which is configured to at least partially displaced the first layer from at least one second layer. The displacement of at least one portion of the first layer from the second layer can occurs solely due to an application of suction by the at least vacuum device on the at least one first layer. The displacement can further occur by an application of a force on the first layer. At least one structural further arrangement can also be used which is configured to damage, without removing, the at least one displaced portion of the first layer. A displacement of adjacent portions of the first layer from the second layer can be performed in the organ by of a translation and/or a rotation of the device along a surface of the structure(s).

### CROSS-REFERENCE TO RELATED APPLICATION(S)

The present application relates to U.S. Patent Application Ser. No. 61/874,686, filed Sep. 6, 2013, the entire disclosure of which is incorporated herein by reference.

### FIELD OF THE DISCLOSURE

The present disclosure relates to exemplary systems, devices, apparatus and methods for providing an endoscopic mucosal therapy, and in particular to exemplary systems, devices, apparatus and methods for treating mucosal diseases (including, e.g., especially mucosal diseases) of the esophagus and the gastrointestinal tract. Such exemplary systems, devices, apparatus and methods can be utilized for an endoscopic treatment of early mucosal cancer and pre-cancerous lesions of the esophagus and gastro-intestinal (GI) tract.

### BACKGROUND INFORMATION

Many diseases originate on superficial tissues such as the mucosal tissues of the gastrointestinal tract. These can include various cancers of the gastrointestinal tract which originate as precancerous mucosal lesions and later invade deeper tissue structures. One strategy for the treatment of these mucosal diseases is to damage the mucosal tissue to a depth sufficient to eliminate the disease, but not sufficiently deep to induce side effects which can include organ perforation or stricture. Such therapy is sometimes called an “ablation” therapy because the mucosal surface is “ablated”, or damaged. A damaged mucosal surface can regrew with a more limited disease extent, or without disease.

The ablation therapy is common in the treatment of early-stage esophageal adenocarcinoma. A common approach is radio-frequency ablation. For example, an endoscopic device is placed adjacent to the mucosal lining of the esophagus. The device contains electrical conductors that provide a localized radio-frequency field which interacts with nearby tissue to heat and thereby affect tissue viability. Because of the radio-frequency field spatial confinement, the damage is limited to the tissues adjacent to the device. This allows mucosal tissues to be targeted while also providing some sparring of deeper tissues such as the submucosa or adventitia. Other methods to provide spatially limited damage, i.e., “ablation”, to the mucosa include laser therapy, photodynamic therapy, and cryotherapy. Multiple devices for delivering these energies to the mucosa have been proposed.

One challenge associated with existing endoscopic mucosal therapy deployments can be that the depth of the therapy is not intrinsically limited to anatomical boundaries, but instead is typically induced to a fixed distance from the device at the time of therapy. In a radio-frequency ablation, for example, a fixed ablation depth is achieved, for example, 400 μm, which does not necessarily correlate to an anatomical landmark, for example, the muscularis mucosa. In addition, the physical thickness of the mucosa can vary significantly between patients and in response to disease state and applied force. For these reasons, among others, existing methods and system for providing an endoscopic mucosal therapy can require multiple treatments sessions to eliminate the lesion, or can be result in damage to tissue anatomical layers that are not intended to be targeted.

An alternative strategy for delineating between the mucosal tissue to be damaged (“ablated”) and that to be sparred is based on differing mechanical properties of the tissue layers. Most gastrointestinal tissues can be arranged in layers. For example, in the esophagus, the most superficial (luminal) layer is the epithelium, followed by the lamina propria, the muscularis mucosa, the submucosa, and finally the muscularis externa (ME). The more superficial layers are not rigidly bound to the other ME layers. Instead, the inner layers can slide a finite distance, can buckle if the ME layers contracts, or can be lifted a finite distance from the ME. This mechanical feature can be used to define a depth boundary in a therapy.

Endoscopic mucosal resection (EMR), for example, relies on the ability of the superficial mucosal layers to be lifted from the deeper layers by fluid injection. In EMR, a fluid such as saline can be injected into the submucosal space, lifting the mucosal surface away from the deeper structures. This lifted tissue is then mechanically resected and removed, and can be analyzed for pathology. EMR is therefore both a diagnostic and a localized therapy, and in the therapy the depth extend is defined in part by how the differing tissue layers are mechanically bound to the others.

When applied as a therapy, EMR is generally used over limited regions. In many endoscopic mucosal therapies such as radio-frequency ablation, it is desired to destroy (“ablate”) large areas of the superficial mucosa.

Accordingly, there is a need for system, device, apparatus and method that can address at least some of the above-described deficiencies, and apply therapies over large areas, and can target the extent of tissue destruction in part based on the mechanical properties of different anatomical tissue layers. Further, EMR resects tissue leaving some bleeding and scarring. There is a need for system, device, apparatus and method that can apply non-surgical (e.g., non-resection) intervention where the extent of tissue therapy is based in part on the mechanical properties of anatomical layers.

### SUMMARY OF EXEMPLARY EMBODIMENTS

Thus, to address at least such issues and/or deficiencies, exemplary embodiments of exemplary systems, devices, apparatus and methods can be provided for an endoscopic mucosal therapy, and in particular to exemplary systems, devices, apparatus and methods for treating mucosal diseases (including, e.g., especially mucosal diseases) of the esophagus and the gastrointestinal tract.

For example, many of these tissues have unique mechanical properties that allow superficial layers to slide and lift over a limited extent relative to deeper tissues. The exemplary systems, devices, apparatus and methods described here can utilize this ability to, e.g., partially separate superficial layers from deeper layers to achieve a therapeutic effect that is largely confined to the superficial tissue. The exemplary systems, devices, apparatus and methods can be operated, for example, by extending a portion of mucosal tissue away from its underlying tissue structures, and applying a local energy, field, force, or other method to damage, destroy, or alter this extended tissue extent. By extending the tissue away from deeper structures, the therapy effect can be better confined to the superficial layers, and that confinement of therapeutic effect can, for example, be based in part on the mechanical properties of differing anatomical layers rather than on a fixed distance.

The exemplary embodiments of exemplary systems, devices, apparatus and methods according to the present disclosure can differs from existing radio-frequency ablation devices and laser thermal therapy devices in that the shape or form of the mucosa is altered such that regions of mucosal tissue are displaced from underlying tissue structures. Further additional differences from an endoscopic mucosal resection can be that the therapeutic effect is not based on surgical excision of tissues but rather on applying a therapeutic effect to tissue in situ, such that that tissue can remain in place for a limited time after the exemplary procedure.

Accordingly, an exemplary embodiment of exemplary systems, devices, apparatus and methods can be provided that can effect at least one first layer of at least one biological structure of an internal organ can be provided. For example, it is possible to utilize at least one structural arrangement which is configured to be inserted into or adjacent to the organ, and including at least one vacuum device which is configured to at least partially displaced the first layer from at least one second layer. The displacement of at least one portion of the first layer from the second layer can occur solely due to an application of suction by the at least vacuum device on the first layer. The displacement can further occur by an application of a force on the first layer. At least one structural further arrangement can also be used which is configured to damage, without removing, the displaced portion of the first layer. A displacement of adjacent portions of the first layer from the second layer can be performed in the organ by of a translation and/or a rotation of the device along a surface of the structure(s).

In a further exemplary embodiment of the present disclosure, the further arrangement can causes the damage to the displaced portion(s) by providing (i) a photo-thermal heating, (ii) a conductive heating, (iii) irreversible electroporation, (iv) radio frequency heating, (v) photo dynamic therapy, and/or (vi) ultrasonic heating. The organ can be (i) esophagus, (ii) lung, and/or (iii) a gastrointestinal organ. One or more of the arrangements can be configured to be affixed to an end portion of an endoscope, to be conveyed through a port of an endoscope, or to be placed at the site without use of an endosope. The further arrangement can be further configured to damage the displaced portion(s) repeatedly more than once. The damage of the displaced portion can avoid an extensive damage of the second layer. For example, after the displaced portion is damaged, such displaced portion can be returned to be adjacent to its original position.

According to yet another exemplary embodiment, the organ can be a luminal organ, and the device can cause a continuous damage to the displaced portion for at least 360°. The arrangement can be further configured to measure a temperature of the displaced portion(s).

These and other objects, features and advantages of the present disclosure will become apparent upon reading the following detailed description of exemplary embodiments of the present disclosure, when taken in conjunction with the appended drawings and appended claims.

### BRIEF DESCRIPTION OF THE DRAWINGS

Further objects, features and advantages of the invention will become apparent from the following detailed description taken in conjunction with the accompanying figures showing illustrative embodiments of the present disclosure, in which:

FIG. 1 is an illustration of exemplary anatomical layers of the esophagus;

FIG. 2 is an exemplary cross-sectional view of a conventional radio-frequency therapy device in an exemplary use;

FIG. 3 are exemplary cross-sectionals view of a therapy device/system/apparatus according to an exemplary embodiment of the present disclosure in use;

FIG. 4 is an exemplary cross-sectional view of the exemplary therapy device/system/apparatus of FIG. 3 during a use of a vacuum suction;

FIG. 5 is an exemplary cross-sectional view of the therapy device/system/apparatus according to another exemplary embodiment of the present disclosure that uses an irreversible electroporation;

FIG. 6 are exemplary views of an exemplary cross-sectional view of the therapy device/system/apparatus according to a further another exemplary embodiment of the present disclosure that facilitates treatment over large areas by sliding or rolling the device across the tissue;

FIG. 7 is an exemplary cross-sectional view of the therapy device/system/apparatus according to additional another exemplary embodiment of the present disclosure that uses rollers to deform the mucosal tissue;

FIG. 8 are exemplary illustrations of the therapy device/system/apparatus according to still further another exemplary embodiment, in operation, for deforming mucosal tissue based on an adherence along three lines;

FIG. 9 are exemplary illustrations of the therapy device/system/apparatus according to additional another exemplary embodiment of the present disclosure for deforming a mucosal tissue based on application of a tape or structure to the tissue, and a coupling of that tape or structure to the exemplary therapy device/system/apparatus;

FIG. 10 is an exemplary cross-sectional view of the therapy device/system/apparatus according to yet another exemplary embodiment of the present disclosure using light or other electro-magnetic radiation to achieve therapeutic effects;

FIG. 11 is an exemplary cross-sectional view of the therapy device/system/apparatus according to a further exemplary embodiment of the present disclosure that uses heaters to achieve therapeutic effects;

FIG. 12 is an exemplary cross-sectional view of the therapy device/system/apparatus according to a still further another exemplary embodiment of the present disclosure which uses light activatable drugs to achieve therapeutic effects;

FIG. 13 is an exemplary schematic of a therapy device using ultrasound transducers to achieve therapeutic effects;

FIG. 14 is an exemplary cross-sectional view of the exemplary therapy device/system/apparatus as shown any of the figures herein affixed to an endoscope;

FIG. 15 are various exemplary diagrams of the exemplary therapy device/system/apparatus as shown in, e.g., any of the figures which is configured to apply therapy across a 360 degree area;

FIG. 16 is an exemplary assembled and exploded three-dimensional view of the device/system/apparatus according to an exemplary embodiment of the present disclosure that uses a heated element to induce thermal injury; and

FIG. 17 is an exemplary cross-sectional view of the device/system/apparatus shown in FIG. 16.

Throughout the drawings, the same reference numerals and characters, if any and unless otherwise stated, are used to denote like features, elements, components, or portions of the illustrated embodiments. Moreover, while the subject disclosure will now be described in detail with reference to the drawings, it is done so in connection with the illustrative embodiments. It is intended that changes and modifications can be made to the described exemplary embodiments without departing from the true scope and spirit of the subject disclosure and the appended claims.

### DETAILED DESCRIPTION OF EXEMPLARY EMBODIMENTS

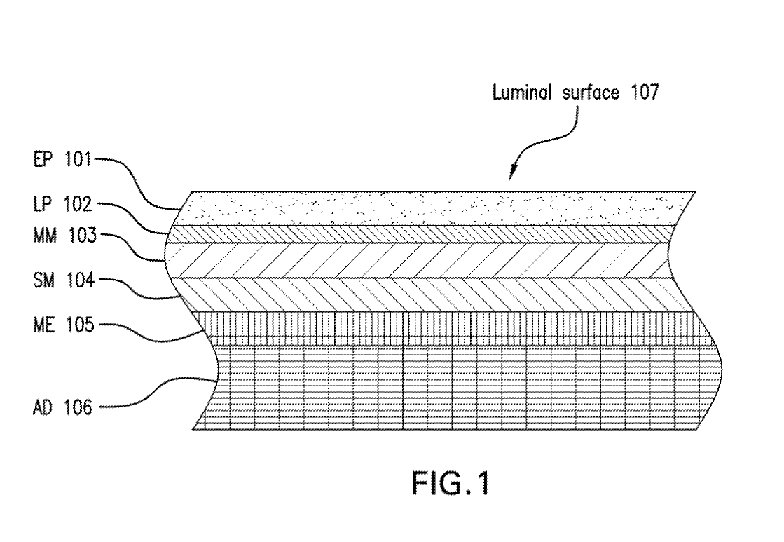

An exemplary layering architecture of a mucosal or other tissue is illustrated in FIG. 1. As shown therein, the luminal surface 107 is defined by the epithelium (EP) 101. The deeper structures are in order of increasing depth from the luminal surface: the lamina propria (LP) 102, the muscularis mucosa (MM) 103, the submucosa (SM) 104, the muscularis externa (ME) 105, and the adventitia (AD) 106. This exemplary arrangement of tissue layers corresponds to that of the normal human esophagus, and is illustrated to be used in connection with the descriptions of exemplary embodiments described hereafter. It should be understood that the exemplary embodiments of the present disclosure described herein can be applied to tissues of different architectural structures, anatomies, and/or locations.

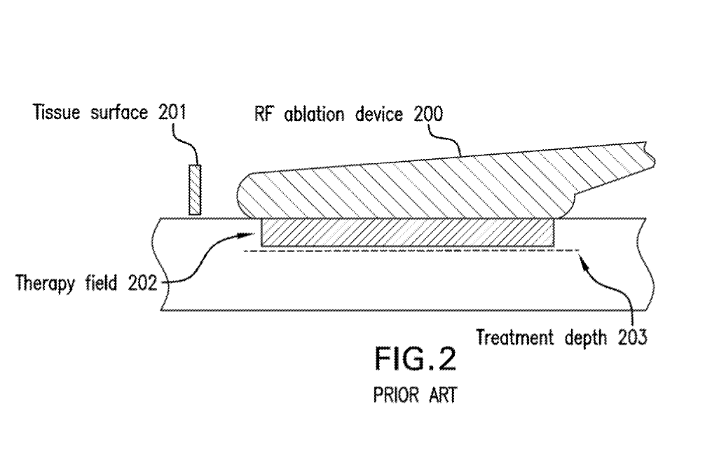

A cross-sectional view of a conventional radio-frequency therapy device 200 in operation is presented in FIG. 2. As shown in FIG. 2, the therapy device 200 is placed in contact with the mucosal tissue surface 201. This device can be a paddle affixed to an endoscope, or a balloon-based therapy device. The device 200 defines a therapeutic field 202 from the device surface to a location 203 within the tissue. For the radio-frequency ablation, this therapeutic field 202 is radio-frequency excitation that ablates the tissue through thermal effects. The ablated or treated tissue can then be mechanically scraped from the organ wall, or can be left to be replaced during healing and new tissue growth. Because the anatomy is not known, the therapeutic depth can be located superficial to a specific anatomical layer or deeper than a specific anatomic layer. As a result, it is likely difficult to target anatomical layers using the conventional device 200. This can lead to an insufficient treatment depth, or to over-treatment (treatment to a larger depth than is needed and or is safe) which can produce side-effects such as stricture or esophageal perforation.

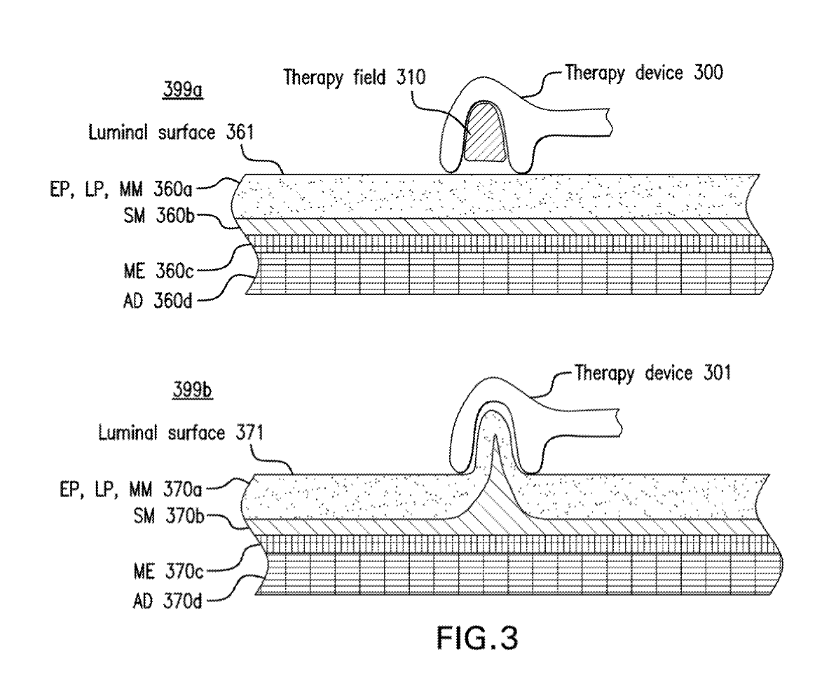

FIG. 3 shows exemplary cross-sectional views of a therapy device (300, e.g., which can also include or be referred to a system and/or an apparatus) according to an exemplary embodiment of the present disclosure in an exemplary use thereof. The initial application of the exemplary device 300 is illustrated in section 300a of FIG. 3. For example, the exemplary device 300 can be placed in contact with the tissue surface 361. The device can be configured to apply a therapy field 361 that is at least partially recessed into the exemplary device. As shown in section 300b of FIG. 3, via a mechanical, vacuum, or other procedure or configuration, the exemplary device 300 can deform the tissue such that superficial layers are brought into the recessed region of the device 301. Here, the layers 370a and 370b are raised into the recessed region where the therapy field 310 is located. The therapy field can then be activated achieving local treatment that is at least partially confined to the tissue layers which are raised into the device. Deeper tissue structures 370c and 370d are at least partially sparred from damage. When the device 300 is removed, the tissue (including the treated tissue) can approximately return to its nominal shape and the treated tissue can, for example, become necrotic and be replaced by new tissue growth over time.

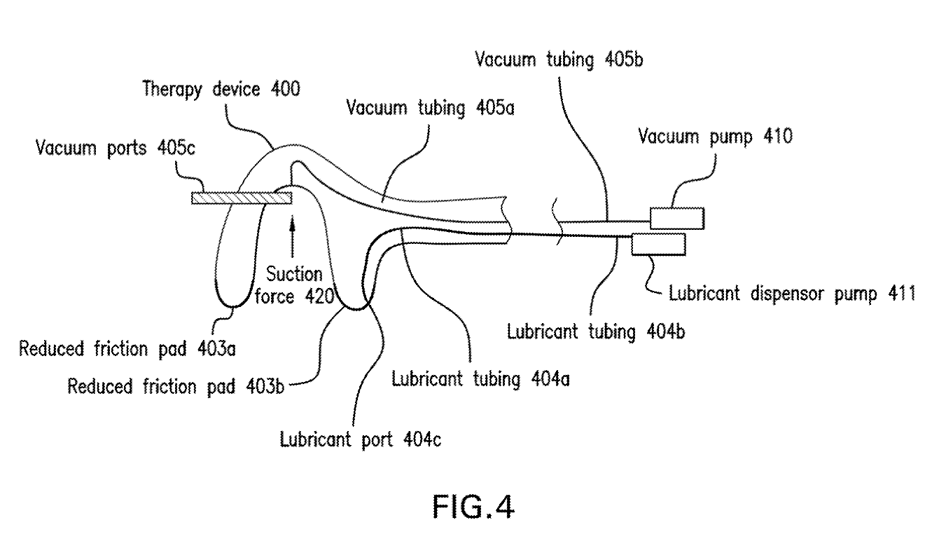

Multiple exemplary methods, configurations, systems, apparatus, devices and/or designs can be provided for extending the mucosa above and away from deeper tissues, as is illustrated in FIG. 3. In one exemplary embodiment of the device/system/apparatus shown in FIG. 4, vacuum (negative) pressure can be used to pull the tissue into a shaped device 400 through a vacuum force 420. The exemplary device 400 can optionally be configured to have regions of limited frictional force (403a, 403b) to allow mucosal tissue to slide laterally. These exemplary regions can be constructed for example by use of materials with reduced friction. such as, e.g., Teflon. The exemplary device 400 can further be configured to have tubing or other conduits 404a, 404b to apply a lubricating material, such as glycerol or water, to enhance tissue sliding. This can be achieved, for example, through a port 404c on the device 400. A lubricant dispenser 411 can be located at a separate location. A vacuum or suction pump 410 can also be located at a location separate from the exemplary therapy device 400. Vacuum can be transmitted to the device 400 through tubing and/or other conduits 405a, 405b, and interface with the tissue through a vacuum port 405c.

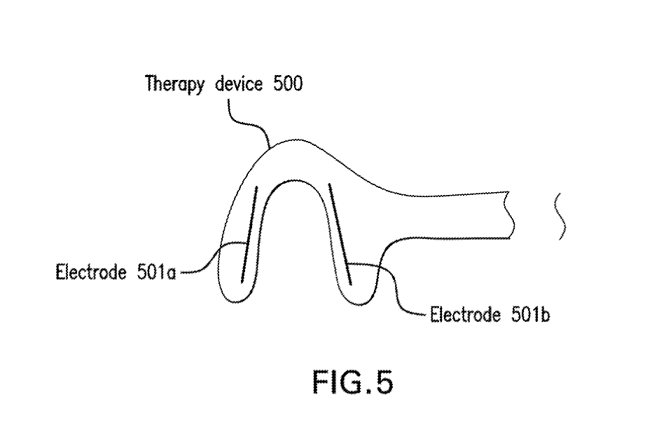

Multiple methods, configuration, systems, apparatus, devices and/or designs can be provided for inducing a therapeutic effect on the mucosal tissue. In one exemplary embodiment of the device/system/apparatus shown in FIG. 5, the device 500 can include two electrodes 501a, 501b for performing an irreversible electroporation (IRE) on the tissue. For example, high-voltage pulses are used to damage the tissue located between the electrodes 501a, 501b through in part non thermal effects. IRE can have the advantage that tissue healing is generally improved after treatment and scarring is minimized relative to thermal damage.

IRE is a mechanism that is different from Radio-Frequency Ablation. For example, in IRE, the tissue damage does not result from thermal injury, and instead from electric-field induced pores in the cell membranes. For example, as shown in FIG. 5, a voltage field can be generated between electrodes 501a, 501b with a magnitude between about 100 and 10,000 kV/cm, and can be delivered as a sequence of pulses with each pulse of duration 1 microsecond to 10 ms and delivered at a repetition frequency of 1 Hz to 100 kHZ. The pulse repetition frequency can be reduced as needed to limit heating effects. Exemplary repetition rates of 1-10 Hz can be common, and higher repetition rates can be supported in this exemplary device Such exemplary device 500 can also be configured to cool the tissue to avoid thermal damage by inclusion of for example a thermoelectric cooler or circulating chilled liquid. This cooling can facilitate a use of higher repetition rates. In this exemplary device, the pulses can be delivered continuously and tissue heating within the therapy region can be reduced by the combination of active cooling and translation of the device.

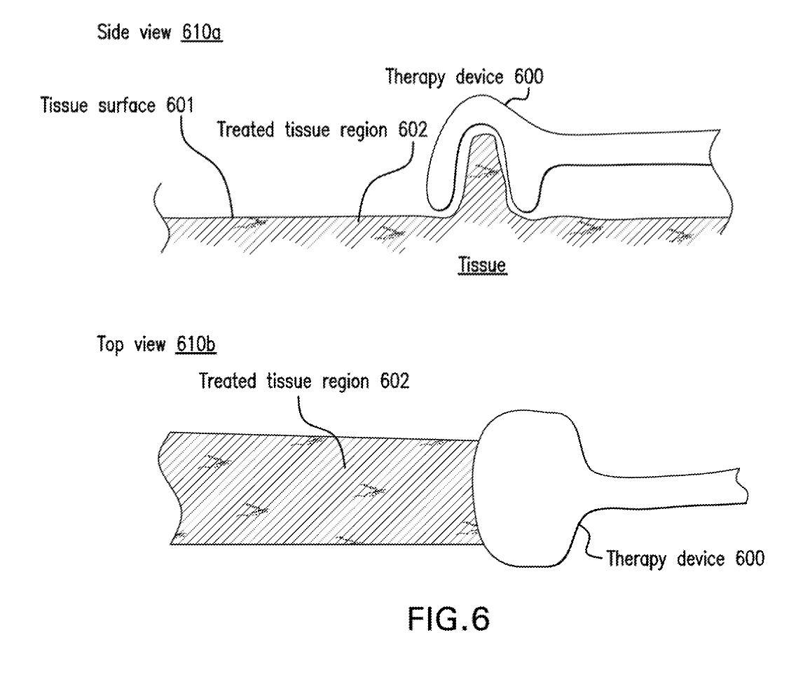

Further multiple methods, configuration, systems, apparatus, devices and/or designs can be provided for applying this therapy over a large field. In on one exemplary embodiment of the device/system/apparatus shown in FIG. 6, the device 600 can be configured, structured and/or sized to allow it to slide along the mucosal surface 601 such that mucosal superficial tissues are pulled into the therapy device along the sliding path and a large area or long stripe 602 of therapeutic effect is induced. In certain exemplary endoscope configurations according to various exemplary embodiments of the present disclosure, this exemplary device 600 can, for example, be attached to the endoscope and can be pulled proximally by the endoscopist through a proximal retraction of the endoscope. When the exemplary device 600 is configured to provide a non-destructive therapeutic effect that does not immediately remove tissue, an exemplary advantage of this exemplary therapy device 600 is that the same tissue locations can be treated repeatedly without significantly extending the therapy depth. For example, if IRE is used, re-treatment of the same tissue will reapply fields to already treated areas and potentially increase therapeutic efficacy of that tissue but it will not significantly extend therapeutic effect deeper to tissue that should be protected. This re-treatment can facilitate large areas of the esophagus to be treated sequentially using for example multiple treatment stripes 602 that can be overlapped to facilitate and/or ensure full coverage of the therapy with a reduced risk of over-treatment. To improve the uniformity of treatment, the exemplary device can be configured and/or structured so as to be retracted at, e.g., a nearly constant velocity by use of a linear motorized stage.

### Further Exemplary Embodiments for Deforming the Mucosal Tissue

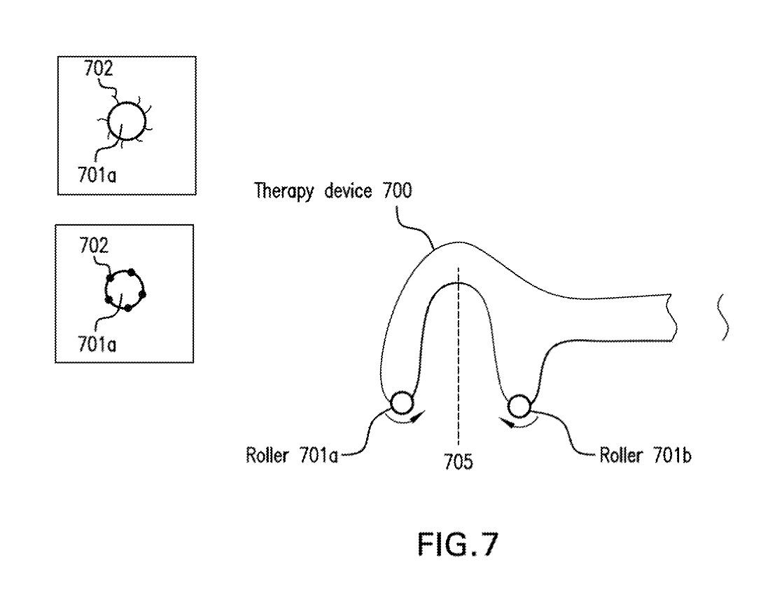

According to other exemplary embodiments of methods, configurations, systems, apparatus, devices and/or designs of the present disclosure shown in FIG. 7, mechanical rotors or roller 701a, 701b can be used to buckle the mucosa through lateral forces. These rotors/rollers 701a, 701b can be configured to contact tissue and push it toward a centerline 705 of the exemplary therapy device 700. This can be done, for example, through spikes 702 on the rotors/rollers 701a, 701b, through a surface preparation and/or suction ports 703, among others. This exemplary configuration can be combined with vacuum configurations and/or procedures described above in connection with the exemplary embodiment shown in FIG. 4. Further, according to another exemplary embodiment, a single rotor/roller can be used, for example, the rotor/roller 701b, and instead of the second rotor 701a. Thus, the exemplary device 700 can be configured to have increased tissue friction such that the lateral force provided by the first rotor/roller 701b would buckle tissue into the cavity of the exemplary therapy device 700. The area configured to have increased tissue friction can be implemented, for example, by surface texturing, by vacuum/suction ports, or by extended spikes similar to those of the spikes 702.

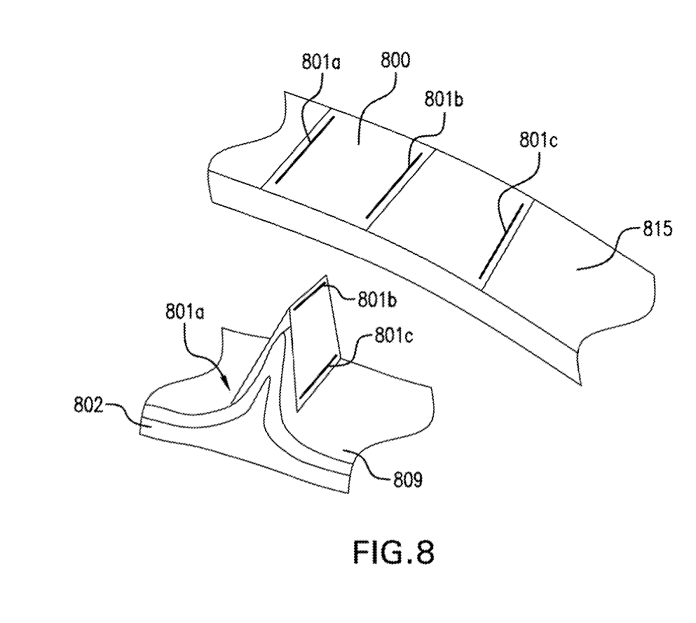

According to another exemplary embodiments of methods, configurations, systems, apparatus, devices and/or designs of the present disclosure shown in FIG. 8, the device 800 can be configured to affix to the mucosal tissue in, for example, three lines 801a, 801b, 801c, and can be configured, structured and/or sized to be largely flat to align each of these lines 801a, 801b, 801c to a flat mucosa surface 815. The exemplary device 800 can be configured to fold such that the tissue affixed to the line 801b is raised, and the tissue affixed to the lines 801a, 801c is laterally moved toward the device center, thus providing a bucking force bringing superficial tissues 809 above deeper tissues 802. The affixing regions or lines 801a, 801b, 801c of the device 800 can operate by vacuum, by adhesives, or by protruding needles/hooks.

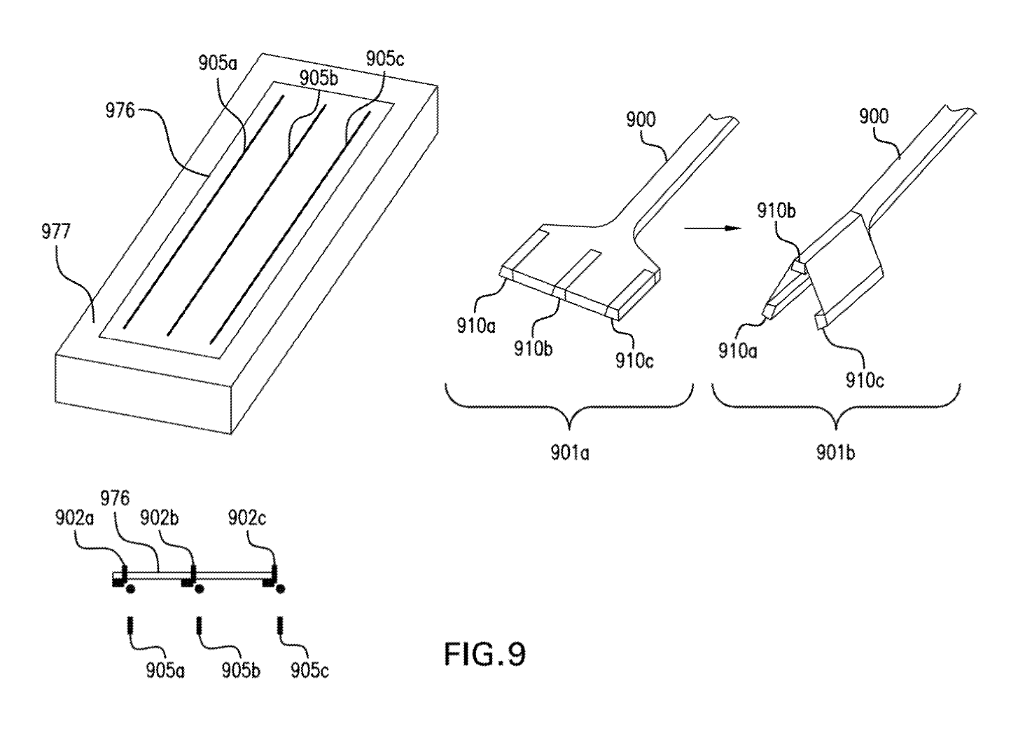

In still another exemplary embodiments of methods, configurations, systems, apparatus, devices and/or designs according to the present disclosure shown in FIG. 9, a tape 976 comprising three adhesive lines 905a, 905b, 905c can be affixed to a tissue surface 977 using, for example, adhesives or mechanical needles/hoods. This tape 976 can be configured or provided with rails 902a, 902b, 902c that can interface with mechanical mounts 910a, 910b, 910c on the endoscopic device 900, as shown in a configuration 901a. The exemplary endoscopic device 900 can then be configured to collapse the tissue by movement of the rails 902a, 902b, 902c to achieve the mucosal tissue separation, as provided in a configuration 901b. In this exemplary design, the long regions can be treated by pulling the device backward along the respective rail 902a, 902b, 902c.

### Further Embodiments

### For Treatment of Deformed Tissues

The exemplary embodiments of systems, devices, apparatus and methods according to the present disclosure can be provided for inducing a therapeutic effect on tissue within the deforming devices described previously. In one exemplary embodiment shown in FIG. 6, IRE can be used to alter cell viability, as described herein. For example, using a similar exemplary design as understood by those having ordinary skill in the art after reviewing the present disclosure, RF ablation of the tissue can be achieved by adjusting the electrical excitation frequency and amplitude.

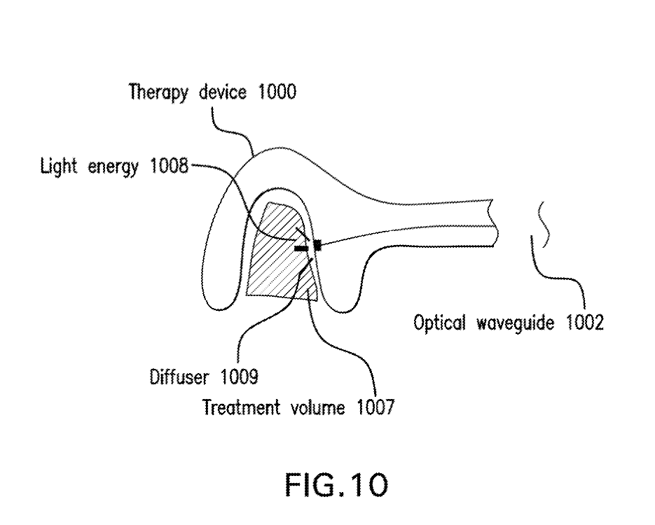

According to yet further exemplary embodiments of methods, configurations, systems, apparatus, devices and/or designs according to the present disclosure shown in FIG. 10, light or other electro-magnetic radiation can be delivered locally to the a treatment volume 1007. Such light or radiation delivery can be achieved, for example, by optical waveguides 1002 that can convey light energy or other radiation 1008 from the console (not shown) to the exemplary device 1000. Multiple optical waveguides can be used to achieve a more uniform light dose to the treatment volume. Such light or radiation light 1008 can be propagated into the mucosal tissue, and its absorption by mucosal tissue can lead to localized heating and thermal damage. For example, light or other radiation with wavelengths between about 1400 nm and 2300 nm can preferably be used to thermally heat tissue. Alternatively, light or other radiation can be used with exemplary wavelengths matched to blood absorption to target blood vessels for therapeutic effect also. The exemplary device 1000 can be specifically configured to prevent light leakage to deeper tissues by adjusting the optical properties of the device and by choosing wavelengths with limited penetration depths in tissue. For example, light/radiation wavelengths that have higher water absorption will propagate shorter distances in tissue and the resultant heating would remain more confined. The light/radiation interface to the treatment volume 1007 can optionally include a diffuser 1009 to spatially disperse density of the light/radiation energy 1008 before entering the tissue.

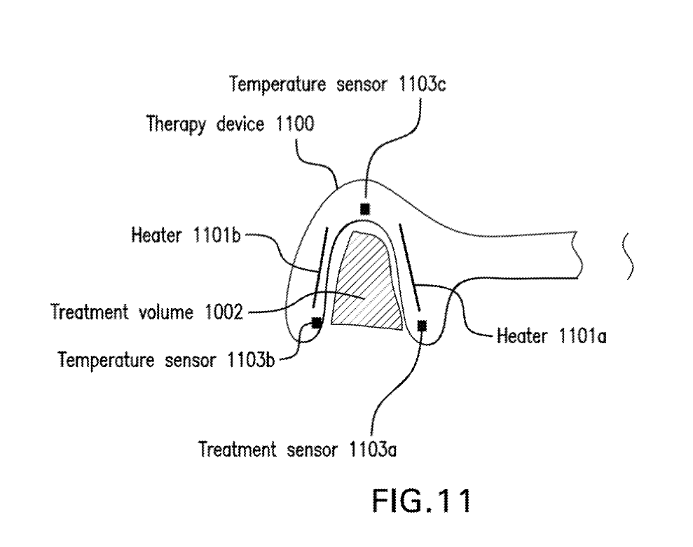

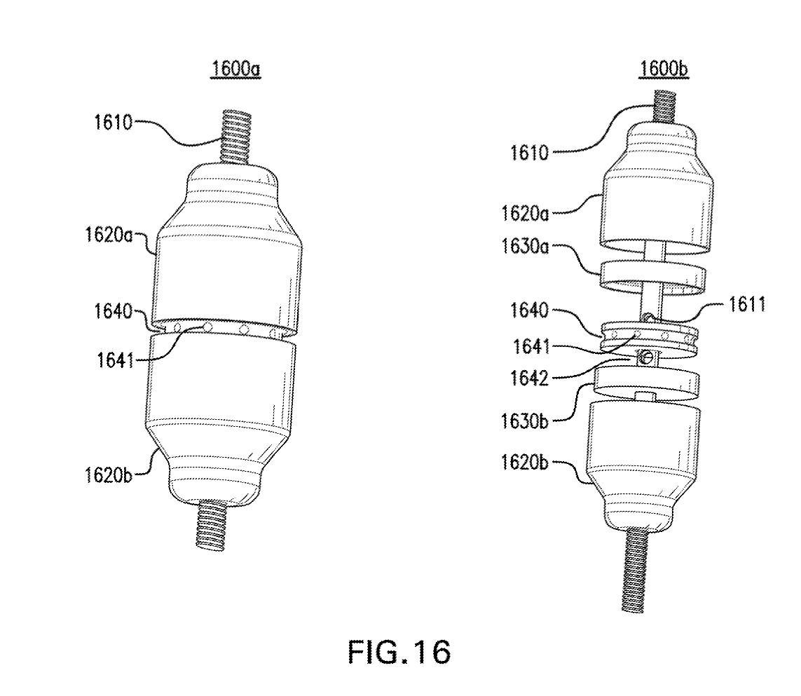

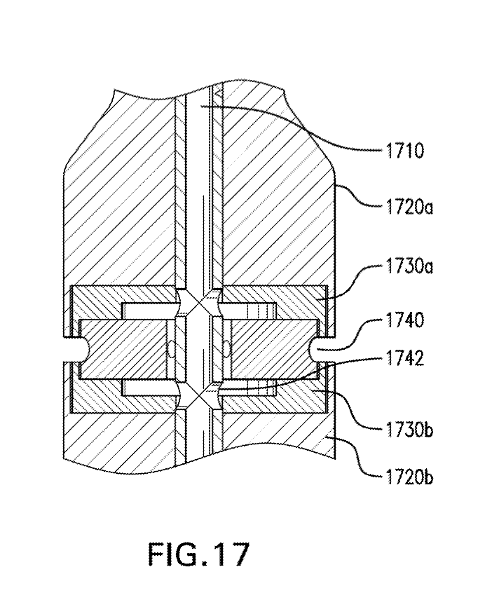

In still further exemplary embodiments of methods, configurations, systems, apparatus, devices and/or designs according to the present disclosure shown in FIG. 11, heaters 1101a, 1101b, such as, e.g., resistive electrical heaters, can be included into or be connected to the exemplary device 1100 to heat a treatment volume 1102 locally. Alternatively, these heaters 1101a, 1101b can be replaced with coolers, such as, e.g., thermoelectric coolers to cool tissue. Temperature sensors 1103a, 1103b, 1103c can be incorporated into or connected to the device 1100 to monitor the tissue temperature. An illustration of another exemplary embodiment of the configuration, system, apparatus, device and/or designs according to the present disclosure is shown in FIG. 16. In this illustration, e.g., an assembled device 1600a is provided alongside its exploded view 1600b. The exemplary device illustrated in FIG. 16 can include a center shaft 1610 that can be hollow, and can conveys a vacuum pressure and houses electrical wires, as well as provided mechanical interface to the console. A first and second piece 1620a can support the organ, and can optionally provide heat sinking or active cooling. A piece 1640 can be heated with integrated heaters (not shown) to a high temperature, for example, 90 degrees Celsius. Two thermal insulating pieces 1630a, 1630b separate the heat sink pieces 1620a, 1620b from the heated piece 1640. These insulating pieces 1630a, 1630b can be made form a plastic such as Teflon. The heated piece 1640 can include radial holes 1641 to convey vacuum pressure, and the shaft 1610 can include holes 1611 to convey vacuum pressure. When assembled, a grove is formed within which therapy can be performed, as shown in FIG. 17. The grove can be formed outside the outer diameter of the heated piece 1740 and can be, for example, between about 0.5 mm to 2 mm deep and 0.5 mm to 2 mm wide (or possibly 1.5 mm deep 1.5 mm wide).

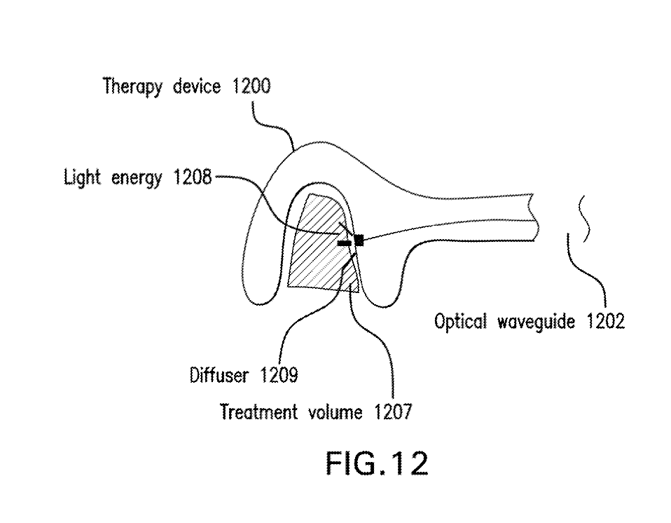

According to other exemplary embodiments of methods, configurations, systems, apparatus, devices and/or designs of the present disclosure illustrated in FIG. 12, light emitters 1207, 1209 analogous or similar to those shown in FIG. 10 located within or connected to the exemplary device 1200 can be used to initiate photodynamic therapy or photoactivatable therapy within the treatment volume 1207 using a photosensitizer drug that was previously delivered to the tissue either topically or systemically. Light or other radiation delivery can be performed using optical waveguides 1202.

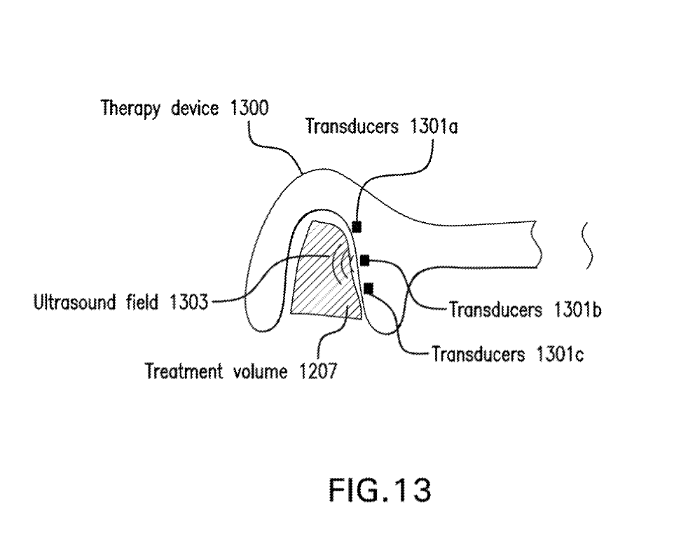

In still other exemplary embodiments of methods, configurations, systems, apparatus, devices and/or designs according to the present disclosure illustrated in FIG. 13, ultrasonic transducers 1301a, 1301b, 1301c located within or connected to the exemplary device 1300 can be used to provide ultrasonic heating or ultrasonic damage to tissue 1302. Using such ultrasonic transducers 1301a, 1301b, 1301c can be used to form an ultrasonic field 1303 in the tissue 1302.

### EMBODIMENT

### For Integration with Endoscopes and Large Area Treatment

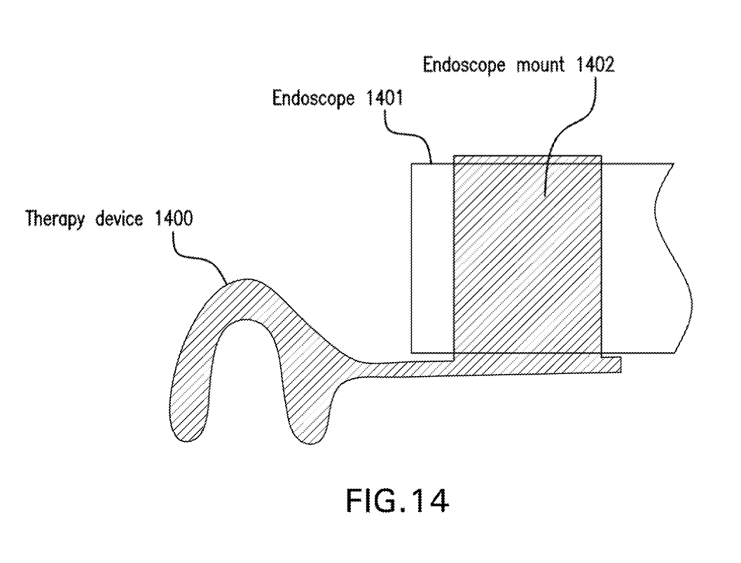

Various exemplary methods or devices to endoscopically deploy these exemplary therapy devices can be used. According to exemplary embodiments of the configurations, systems, apparatus, devices and/or designs of the present disclosure illustrated in FIG. 14, the therapy device 1400 can be affixed or otherwise connected to an endoscope 1401 using a mounting 1402, and can hang as a paddle that occupies a portion of the endoscopes' circumferential view, for example 90 degrees. This exemplary paddle can extend into the endoscope's imaging field, as shown in FIG. 14, such that the treated tissue can be visualized, or can be drawn proximally to move it outside the imaging field, or alternatively can be designed or configured to move dynamically into and out of the imaging field, as needed.

In another exemplary embodiment of the present disclosure, the exemplary therapy device can be configured to alter the treated tissue such that treated regions can be visually or endoscopically differentiated from untreated regions. For some exemplary methods of therapy, such as thermal injury, this marking of the treated tissue can be related to the therapeutic effect directly. Alternatively, a more limited thermal injury can be induced by light/radiation and/or by the heaters to mark the treatment region or to mark the borders of treated regions. For example, in the exemplary device/system/apparatus shown in FIG. 5, resistive heaters 1101a, 1101b—as shown in FIG. 11—can be included to induce visible burn marks of the treated tissue area, e.g., to thermally mark the treatment region. Other marking methods include injection or adherence of ink or dye into or onto the tissue, or induction of visible marks from cutting, wounding, needle puncturing the tissue. Within the context of IRE, for example, ink could be moved into the tissue through the IRE process allowing areas treated with IRE to be marked.

In a further exemplary embodiment of the present disclosure, the device/system/apparatus can be translated along the tissue to achieve larger areas—as depicted in FIG. 6. The exemplary device can be configured to monitor this displacement such that feedback can be given to the treatment personnel or directly to the device to control the application of the therapy, i.e., to stop treatment once the sliding motion has stopped and to resume once the sliding motion resumes, or to adjust the dosimetry based on the sliding velocity of the device. This exemplary monitoring can be performed using, for example, gyroscopes, endoscopic camera sensors, RF positional sensors, and/or sensors located with the device that can read out, for example, the treated tissue temperature. In addition, exemplary motorized mechanisms for moving the device in the treatment direction can be used such that at least nearly or possibly exactly constant velocity can be achieved.

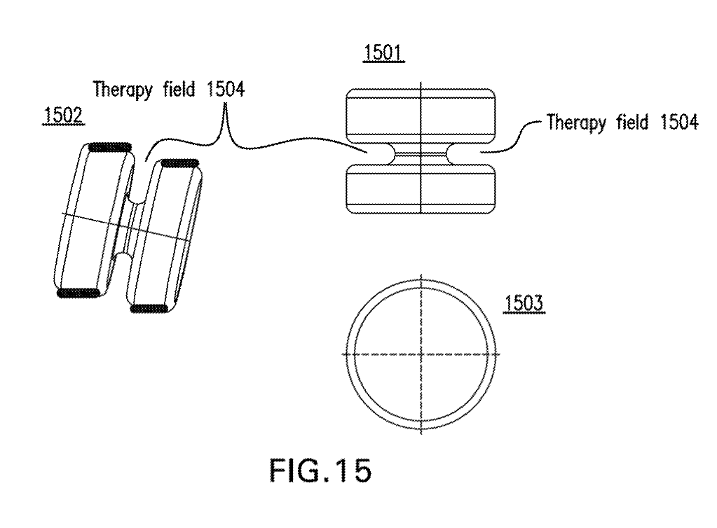

In yet another exemplary embodiment of the present disclosure, the exemplary device/system/apparatus can be designed and/or configured to treat simultaneously approximately 360 degrees of a luminal organ mucosa, as depicted in FIG. 15. As shown in FIG. 15, an exemplary device 1500 provided for deforming the tissue can have a circumferential design 1501 that can treat an area for 360 degrees by providing an appropriate therapy fields 1502, 1504, and for a corresponding cross-section 1503 associated with the design 1501. Exemplary designs and/or configurations achieving treatment less than 360 degrees can also be constructed.

According to yet a further exemplary embodiment of the present disclosure, the exemplary device can be configured to include sensing, imaging and/or spectroscopy such that properties of the displaced tissue can be measured to determine in real time if treatment should be applied. This diagnostic for sensing, imaging, or spectroscopy arrangements can include, for example, optical coherence tomography imaging, camera imaging, light absorption or reflection spectroscopy, confocal microscopy, or Raman spectroscopy.

In yet another exemplary embodiment of the present disclosure, the exemplary device/system/apparatus can include multiple recessed portions 310, as shown in FIG. 3, which can be positioned along the device. For example, during a translation or rotation of the exemplary device/system/apparatus, a tissue portion can be first treated by the first recessed portion, and later re-treated by the second recessed portion.

According to still another exemplary embodiment of the present disclosure, the delivery of energy to and/or by the exemplary device/system/apparatus can be facilitated in a continuous wave operational mode, and/or can be pulsed. For example, light (or other electromagnetic radiation) provided by the diffuser 1009 shown in FIG. 10 can be pulsed at a repetition rate, for example, in a range of between about 0.1 Hz and 1 kHZ, as well as outside such range.

In yet another exemplary embodiment of the present disclosure, the device/system/apparatus can be configured and/or structured to provide heat sinking from regions adjacent to the energy delivery, for example the regions 403a, 403b can be constructed substantially from metal such that the portions of tissue adjacent to these regions remain at lower temperatures. These exemplary regions 403a, 403b can also be actively cooled by for example a thermoelectric cooler or circulating chilled liquid within the device.

The foregoing merely illustrates the principles of the disclosure. Various modifications and alterations to the described embodiments will be apparent to those skilled in the art in view of the teachings herein. Indeed, the arrangements, systems and methods according to the exemplary embodiments of the present disclosure can be used with and/or implement any OCT system, OFDI system, SD-OCT system or other imaging systems, and for example with those described in International Patent Application PCT/US2004/029148, filed Sep. 8, 2004 which published as International Patent Publication No. WO 2005/047813 on May 26, 2005, U.S. patent application Ser. No. 11/266,779, filed Nov. 2, 2005 which published as U.S. Patent Publication No. 2006/0093276 on May 4, 2006, and U.S. patent application Ser. No. 10/501,276, filed Jul. 9, 2004 which published as U.S. Patent Publication No. 2005/0018201 on Jan. 27, 2005, and U.S. Patent Publication No. 2002/0122246, published on May 9, 2002, the disclosures of which are incorporated by reference herein in their entireties. It will thus be appreciated that those skilled in the art will be able to devise numerous systems, arrangements and methods which, although not explicitly shown or described herein, embody the principles of the disclosure and are thus within the spirit and scope of the present disclosure. It should be understood that the exemplary procedures described herein can be stored on any computer accessible medium, including a hard drive, RAM, ROM, removable disks, CD-ROM, memory sticks, etc., and executed by a processing arrangement and/or computing arrangement which can be and/or include a hardware processors, microprocessor, mini, macro, mainframe, etc., including a plurality and/or combination thereof. In addition, certain terms used in the present disclosure, including the specification, drawings and claims thereof, can be used synonymously in certain instances, including, but not limited to, e.g., data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it can be explicitly incorporated herein in its entirety. All publications referenced herein can be incorporated herein by reference in their entireties.

1. An apparatus for effecting at least one first layer of at least one biological structure of an internal organ, comprising:
at least one structural arrangement which is configured to be inserted into or adjacent to the organ, and including at least one vacuum device which is configured to at least partially displace the at least one first layer from at least one second layer, wherein the displacement of at least one portion of the first layer from the second layer occurs solely due to an application of suction by the at least vacuum device on the at least one first layer.

2. The apparatus according to claim 1, wherein the displacement further occurs by at least one of (a) an application of a force on the at least one first layer or (b) in the organ by at least one of a translation or a rotation of the at least one device along a surface of the at least one structure, further comprising:
at least one structural further arrangement which is configured to damage, without removing, the at least one displaced portion of the first layer.

3. The apparatus according to claim 2, wherein the further arrangement causes the damage to the at least one displaced portion at least one of:
(a) by providing at least one of (i) a photo-thermal heating, (ii) a conductive heating, (iii) irreversible electroporation, (iv) radio frequency heating, (v) photo dynamic therapy, or (vi) ultrasonic heating, or
(b) repeatedly more than once.

4. The apparatus according to claim 1, wherein the organ is at least one of (i) esophagus, (ii) lung, or (iii) a gastrointestinal organ.

5. The apparatus according to claim 2, wherein at least one of:
(a) at least one of the arrangement or the further arrangement are configured to be affixed to an end portion of an endoscope
(b) the damage of the at least one displaced portion avoids an extensive damage of the second layer,
(c) after the at least one displaced portion is damaged, the at least one displaced portion is returned to be adjacent to its original position, or
(d) the organ is a luminal organ, and wherein the at least one device causes continuous damage to the at least one displaced portion for at least 360.

6-9. (canceled)

10. The apparatus according to claim 1, wherein the arrangement is further configured to measure a temperature of the at least one displaced portion.

11. (canceled)

12. A method for effecting at least one first layer of at least one biological structure of an internal organ, comprising:
inserting a structural arrangement into or adjacent to the organ, the structural arrangement including at least one vacuum device which is configured to at least partially displaced the at least one first layer from at least one second layer; and
facilitating the displacement of at least one portion of the first layer from the second layer solely due to an application of suction by the at least vacuum device on the at least one first layer.

13. An apparatus for effecting at least one first layer of at least one biological structure of an internal organ, comprising:
at least one structural first arrangement which is configured to be inserted into or adjacent to the organ, and including at least one device which is configured to at least partially displace at least one portion of the at least one first layer from at least one second layer by an application of a force on the at least one first layer; and
at least one structural second arrangement which is configured to damage, without removing, the at least one displaced portion of the first layer.

14. The apparatus according to claim 13, wherein the displacement of at least one portion of the first layer from the second layer occurs solely due to an application of suction by the at least vacuum device on the at least one first layer.

15. The apparatus according to claim 13, wherein the second arrangement at least one of:
(a) causes the damage to the at least one displaced portion by providing at least one of (i) a photo-thermal heating, (ii) a conductive heating, (iii) irreversible electroporation, (iv) radio frequency heating, (v) photo dynamic therapy, or (vi) ultrasonic heating, or
(b) damages the at least one displaced portion repeatedly more than once.

16. The apparatus according, to claim 13, wherein the organ is at least one of (i) esophagus, (ii) lung, or (iii) a gastrointestinal organ.

17. The apparatus according to claim 13, wherein at least one of:
(a) at least one of the first arrangement or the second arrangement are configured to be affixed to an end portion of an endoscope,
(b) the damage of the at least one displaced portion avoids an extensive damage of the second layer,
(c) after the at least one displaced portion is damaged, the at least one displaced portion is returned to be adjacent to its original position, or
(d) the organ is a luminal organ, and wherein the at least one device causes continuous damage to the at least one displaced portion for at least 360, or
(e) the first arrangement is further configured to measure a temperature of the at least one displaced portion.

18-22. (canceled)

23. The apparatus according to claim 13, wherein the displacement further occurs performed in the organ by at least one of a translation or a rotation of the at least one device along a surface of the at least one structure.

24. A method for effecting at least one first layer of at least one biological structure of an internal organ, comprising:
inserting a structural arrangement into or adjacent to the organ, the structural arrangement including at least one vacuum device which is configured to at least partially displaced the at least one first layer from at least one second layer;
damaging, without removing, the at least one displaced portion of the first layer.

25. An apparatus for effecting at least one first layer of at least one biological structure of an internal organ, comprising:
at least one structural arrangement which is configured to be inserted into or adjacent to the organ, and including at least one device which is configured to at least partially displaced the at least one first layer from at least one second layer, wherein the displacement of adjacent portions of the first layer from the second layer is performed in the organ, and wherein at least one of a translation or a rotation of the at least one device is performed along a surface of the at least one structure.

26. The apparatus according to claim 25, wherein the displacement further occurs by at least one of (a) an application of a force on the at least one first layer or (b) solely due to an application of suction by the at least vacuum device, on the at least one first layer, further comprising:
at least one structural further arrangement which is configured to damage, without removing, the at least one displaced portion of the first layer.

27. The apparatus according to claim 25, wherein the organ is at least one of (i) esophagus, (ii) lung, or (iii) a gastrointestinal organ.

28. The apparatus according to claim 26, wherein the at least one further arrangement causes the damage to the at least one displaced portion by providing at least one of (i) a photo-thermal heating, (ii) a conductive heating, (iii) irreversible, electroporation, (iv) radio frequency heating, (v) photo dynamic therapy, or (vi) ultrasonic heating.

29. The apparatus according to claim 26, wherein at least one of:
(a) at least one of the structural arrangement or the further arrangement are configured to be affixed to an end portion of an endoscope,
(b) the at least one further arrangement is further configured to damage the at least one displaced portion repeatedly more than once,
(c) the damage of the at least one displaced portion avoids an extensive damage of the second layer,
(d) after the at least one displaced portion is damaged, the at least one displaced portion is returned to be adjacent to its original position, or
(e) the organ is a luminal organ and wherein the at least one device causes continuous damage to the at least one displaced portion for at least 360°.

30-33. (canceled)

34. The apparatus according to claim 25, wherein the at least one structural arrangement is further configured to measure a temperature of the at least one displaced portion.

35. (canceled)

36. A method for effecting at least one first layer of at least one biological structure of an internal organ, comprising:
inserting a structural arrangement into or adjacent to the organ, the structural arrangement including at least one vacuum device which is configured to at least partially displaced the at least one first layer from at least one second layer;
facilitating the displacement of at least one portion of the first layer from the second layer in the organ; and
performing at least one of a translation or a rotation of the at least one device along a surface of the at least one structure.
